(19) United States
(12) Patent Application Publication
(10) Pub. No.: US 2022/0354752 A1
(43) Pub. Date: Nov. 10, 2022

(54) NANOFIBRE MATRIX MADE OF NATURAL POLYMERS WITH NATURAL FUNCTIONAL INGREDIENTS FOR COSMETIC PRODUCTS

(71) Applicant: NANOLAYR LIMITED, Auckland (NZ)

(72) Inventors: Bhuvaneswari KANNAN, Auckland (NZ); Iain Cameron HOSIE, Auckland (NZ); Han Yue ZHENG, Auckland (NZ)

(21) Appl. No.: 17/764,773

(22) PCT Filed: Oct. 8, 2020
(86) PCT No.: PCT/NZ2020/050121

§ 371 Date: Mar. 29, 2022

(30) Foreign Application Priority Data
Oct. 8, 2019 (CN) 2019109922500
Oct. 8, 2019 (CN) 2019109923626

(51) Int. Cl. A61K 8/02; A61K 8/73; A61K 8/65; A61Q 19/02; A61K 8/67; A61Q 19/08; A61K 8/60; A61Q 1/04

(52) U.S. Cl. CPC A61K 8/0208; A61K 8/73; A61K 8/65; A61K 8/0212; A61Q 19/02; A61K 8/676; A61K 8/735; A61Q 19/08; A61K 8/602; A61Q 1/04; A61K 2800/432

(57) ABSTRACT

Described herein is a nanofibre matrix comprising 100% natural biopolymers selected from polysaccharides and/or proteins with optional functional ingredients for use as cosmetic products. More specifically, nanofibre matrix manufactured by electrospinning methods comprising a dry sheet without containing any preservatives, surfactants, solvents and crosslinkers. To use nanofibre cosmetic products, simply wet skin and then apply the nanofibre to skin. Functional ingredients may be optionally incorporated within the nanofibre matrix during electrospinning to provide different cosmetic effects. Functional ingredients offer skin benefits such as moisturizing, anti-ageing, wrinkle reduction, brightening, antioxidant and acne treatment. Functional ingredients can be essential oil where it is incorporated into nanofibre matrix to provide scent effect to cosmetic products. Functional ingredients can also be natural dye where it is incorporated in nanofibre matrix to make coloured nanofibres. Coloured nanofibres can be used for makeup products such as blush, eye shadow, eyebrow, nail polish, lipstick, contouring and hair dye product. The nanofibre matrix comprising 100% natural biopolymers with or without functional ingredients can be processed to assorted shapes or easily cut by consumers for using on different areas of skin for different purposes.

### RELATED APPLICATIONS

This application derives priority from Chinese patent application numbers 2019109923626 and 2019109922500 incorporated herein by reference.

### TECHNICAL FIELD

Described herein is a nanofibre matrix comprising 100% natural biopolymers selected from polysaccharides and/or proteins with optional functional ingredients for use as cosmetic products. More specifically, nanofibre matrix manufactured by electrospinning methods comprising a dry sheet without containing any preservatives, surfactants, solvents and crosslinkers. To use nanofibre cosmetic products, simply wet skin and then apply the nanofibre to skin. Functional ingredients may be optionally incorporated within the nanofibre matrix during electrospinning to provide different cosmetic effects. Functional ingredients offer skin benefits such as moisturizing, anti-ageing, wrinkle reduction, brightening, antioxidant and acne treatment. Functional ingredients can be essential oil where it is incorporated into nanofibre matrix to provide scent effect to cosmetic products. Functional ingredients can also be natural dye where it is incorporated in nanofibre matrix to make coloured nanofibres.

Coloured nanofibres can be used for makeup products such as blush, eye shadow, eyebrow, nail polish, lipstick, contouring and hair dye product. The nanofibre matrix comprising 100% natural biopolymers with or without functional ingredients can be processed to assorted shapes or easily cut by consumers for using on different areas of skin for different purposes.

### BACKGROUND ART

In recent years, the cosmetic industry continues to rapidly grow and consumers are willing to spend more on cosmetic products. Consumers are selecting products based on the efficacy, safety, wellness, sustainability, ethics and convenience. Consumers are more willing to buy natural and eco-friendly products, prefer food-standard and cosmetic/GMP certified cosmetic products due to better regulation associated with food grade ingredients, better sustainable processes and less negative side effects.

There is a large variety of cosmetic products in the market. The facial masks category has many different variants such as: sheet mask, gel mask, clay mask and dry mask. The sheet mask is a very common variant in the market. Traditional sheet masks are manufactured by soaking a fabric material with liquid solution. The liquid solution normally contains the active ingredients, viscosity adjuster polymers, water, preservatives, penetration enhancers, solvents and surfactants. For application, traditional sheet mask requires the wet sheet to be left on the skin for about 10 to 30 minutes, removing and discarding the sheet and then rinsing off afterwards. It is not convenient for people to use and is typically applied at home or in a spa or clinic, while in a seated or prone position.

Furthermore, traditional sheet masks must contain preservatives to prevent bacterial growth in a liquid environment. Preservatives such as: parabens, sodium benzoate, potassium sorbate and phenoxyethanol might be harmful to the skin. Preservatives or solvents can possibly lead to irritation, erythema or uncomfortable feelings for those people who have very sensitive skin. More educated customers in the market understand the side effect of these chemicals and try to avoid these ingredients.

If not worn for the allocated time, traditional sheet masks will not provide desired skin benefits; but if worn for too long, the evaporation of water will result in dry skin. It is also difficult to control the dosage and efficiency of ingredients absorbed by skin. Moreover, traditional sheets masks are not ideal for all skin conditions. For example, traditional masks have been identified as being unsuitable for acne-prone skin as the wet environmental conditions encourages bacterial growth, which can make acne problems worse.

The dry mask technique, is an emerging category that avoids preservatives or organic solvents. Most dry masks on the market are manufactured by freeze drying method. Ingredients are maintained in the substrates (supporting fabric). To apply dry mask, dry mask needs to be soaked in water first before applying on to the skin. Although the dry mask overcomes the problems of organic solvents, it still requires long application times, usually still 10-30 minutes. It does not overcome all existing problems in traditional sheet mask. It is still difficult to control the dosage of ingredients absorbed by skin, and usually contains synthetic polymers and crosslinkers, which are still not ideal for sensitive skin.

Electrospinning is a production method which uses high voltage to turn wet polymer solutions into dry nanofibres. Nanofibre facial masks can solve the problems in traditional mask referenced above such as long application time, limited use environmental conditions, uncontrolled dosage, containing unwanted solvent and materials.

CN104703507B patent discloses a cosmetic film composed of nanofibre produced by electrospinning. The nanofibre layer disclosed in CN104703507B is manufactured by water soluble polymers polyvinyl alcohol and polyvinylpyrrolidone, and also a crosslinking agent.

CN109576817A patent discloses a method to manufacture nanofibre facial masks made by polyvinyl alcohol and sodium alginate. The concentration of polyvinyl alcohol in CN109576817A was between 7%-10% and sodium alginate was between 0.4%-3% in electrospinning solution.

CN109363967A patent discloses a nanofibre facial mask made of 60-80 parts of hyaluronic acid, 10-20 parts of oxidized polyethylene, and 5-10 parts of a bioactive peptide.

These patents mentioned above demonstrated the electrospinning ability of polymers and potential application of nanofibres. However, the produced nanofibre in these patents are made of synthetic polymers or partially made of synthetic polymers. It does not solve the existing problems in traditional masks. It is not suitable for people who have sensitive skin that cannot tolerate synthetic polymers. Nor it is not ideal for people to use if they have environmental concerns about degradation of synthetic plastics and prefer more natural and bio-derived products. Also, the crosslinking agent used in patent CN104703507B is a chemical which is usually not preferred in cosmetic products. Some of synthetic polymers and chemicals can be harmful to the skin if it is used in a long term.

U.S. Pat. No. 9,775,917 patent discloses marine sourced collagen nanofibres with the ability of fast release of active ingredients once the collagen nanofibres are in contact with moisture. However, the nanofibres in this invention are limited to animal based collagen.

From the above, it can be seen that there is a use for a nanofibre composition and related product that overcomes the problem of synthetic polymers and meanwhile overcomes the problems of extended application times, uncontrolled dosage in traditional facial skin masks, or at least provides the public with a useful choice.

It is envisaged that the present invention may overcome these problems and discloses an invention of cosmetic product comprising 100% natural polymers, more specifically, polysaccharides and/or proteins. Functional ingredients like vitamins, fruit/plant/herbal extracts, essential oils and natural dye can be optionally incorporated into the nanofibre matrix to provide advanced skin benefits, colour, scent or flavour.

Further aspects and advantages of the product, composition, methods and uses thereof will become apparent from the ensuing description that is given by way of example only.

### SUMMARY

Described herein is a polysaccharide and/or protein nanofibre matrix with functional ingredients for skin care and cosmetic products. More specifically, a biomaterial-based nanofibre matrix manufactured from a combination of polysaccharides and/or proteins for use in skin care/cosmetic products and manufactured by an electrospinning method. For advanced skin benefits, functional ingredients may be optionally incorporated in the nanofibre matrix during electrospinning and delivered into the skin with the nanofibres. It overcomes the existing problems in traditional cosmetic products. By selecting desired functional ingredients, the nanofibre matrix disclosed in this invention can offer a variety of skin benefits, makeup purpose or scent function. Due to its dry format, it is easily processed into different shapes or manually cut by consumers for various purposes.

In a first aspect there is provided a nanofibre matrix comprising of:

at least one type of polysaccharide; and/or optionally

at least one type of protein

the nanofibre matrix optionally carries with functional active(s) or ingredient(s) for advanced skin care application, wherein the nanofibres are produced from a mixed polysaccharide and/or protein solution with or without the added functional active(s) or ingredient(s) and electrospun into a dry nanofibre non-woven textile such as a dry nanofibre sheet; preferably, the dry nanofibre sheet is processed into different shapes and applied to different areas of skin; and wherein, preferably upon contact with wet skin, the nanofibres are gradually dissolved into the skin and release any actives or sits on the skin to release any actives for advanced therapeutic benefits.

In a second aspect there is provided a cosmetic product in the form of a dry nanofibre matrix with or without added functional active(s) or ingredient(s), wherein the nanofibre matrix is manufactured of 100% natural biopolymers selected from polysaccharides and/or proteins and are electrospun into a dry nanofibre sheet.

In a third aspect there is provided a method of manufacturing a nanofibre matrix as substantially described above.

As above, traditional facial masks have the problems of requiring a long application time, containing synthetic polymers, containing surfactants, containing organic solvents, containing preservatives and also uncontrolled penetration dosage. The advantages of the nanofibre facial mask described in this invention overcomes the problems listed in traditional facial masks. A controlled dosage of ingredients is immediately and efficiently delivered into the skin. The use of nanofibre product is simple and convenient. Nanofibres with or without these functional ingredients are applied onto the wet skin, and immediately absorbed by skin. Nanofibre matrix with or without functional ingredients used for facial mask can overcome the existing deterrents associated with traditional facial masks. Traditional facial masks require a long period of application time, slow penetration, and uncontrolled penetration dosage.

Traditional facial masks also contain synthetic polymers, surfactants, organic solvents, preservatives and other functional additives to stabilize the product from environmental vulnerability such as oxidation or bacterial growth. Nanofibre facial mask described in this invention overcomes these problems. A nanofibre facial mask made of polysaccharides and/or proteins are naturally derived and does not contain any synthetic polymers. Due to its dry format and low water activity, it does not require any preservatives to inhibit the bacterial growth. The nanofibre mask disclosed in this invention does not contain any surfactants and solvent so that it is ideal for people who have sensitive skin. Furthermore, polysaccharides or protein are natural polymers and biodegradable, so it has less environmental impact comparing to synthetic polymers. By selecting food grade polysaccharides and/or proteins to make nanofibre matrix, the resulting products can be considered as ‘edible’ and ‘skin safe’. By selecting plant-based polysaccharides and/or plant-based proteins, it provides users the option of vegan products.

As aforementioned, the mask does not contain any preservatives, surfactants, organic solvents, which will be preferred by people who have sensitive skin. Moreover, the polymer matrix is selected from plant sourced ingredients. It provides users the option of avoiding any animal-based products and also suitable for users who have environmental concerns on the use of synthetic polymers. Furthermore, the use of a dry nanofibre mask allows processing into assorted shapes that include whole facial masks, nose masks, under-eye patches, forehead masks, chin masks, cheek masks, neck masks and a lip mask or the like.

Also, the shape of mask can be decided by users and the users can manually cut the sheet mask into any shapes in order to fit into their requirements. Due to its dry nature, it is easy to cut into any shape. This invention can overcome the problems of current “uni-size” mask which are not always a good fit for the everyday user. Especially for the case of acne treatment, users only need to cut the dry nanofibre into the size which can apply to the infected area without impacting unaffected skin. For a traditional mask, these are often unsuitable for acne skin as a traditional mask contains liquid and the wet environment can cause bacteria growth. Due to is need to be left on the skin for a long time, bacteria growth is not ideal for acne-prone skin and can make the acne problem worse. In this invention, nanofibre matrix is expanded to a wide range of polysaccharides and proteins including those materials extracted from plant based. This offers the opportunity of manufacturing vegan products. The application time is short, less than 1 minute. The ingredients are in a controlled dosage and are absorbed by skin. It is convenient and suitable for use in any situations, such as in transport, work place, public areas and the like.

Finally, this invention discloses a method to produce an innovative skin care product and overcomes the problems in traditional facial masks. The skin benefit properties can be adjusted by selecting different functional ingredients in the polysaccharide and/or protein nanofibre matrix.

### BRIEF DESCRIPTION OF THE DRAWINGS

Further aspects of the product, composition, methods and uses thereof will become apparent from the following description that is given by way of example only and with reference to the accompanying drawings in which:

FIG. 1 illustrates a nanofibre matrix comprising dextran, pullulan and collagen: (a) SEM image; (b) Diameter distribution;

FIG. 2 illustrates optical coherence tomography graphs with optical coherence tomography signals at different timeframes after the nanofibres are applied onto the skin surface: (a) t=0, t=20 mins and t=30 mins; (b) t=0, t=35 mins, t=45 mins and t=50 mins;

FIG. 3 illustrates a vegan nanofibre matrix comprising dextran, pullulan and fucoidan: (a) SEM image; (b) Diameter distribution;

FIG. 4 shows a table of TNF-alpha concentrations in negative control (non-treated epidermis), positive control (epidermis treated with 0.1% SDS), and a tested sample (epidermis treated with vegan nanofibres and 0.1% SDS), after 24 hours incubation;

FIG. 5 illustrates a graph of melanin concentration in cells only, cells with kojic acid and cells with nanofibres and ethyl ascorbic acid after 96 hours incubation;

FIG. 6 illustrates a graph of elastin concentration in cells only, cells with nanofibre matrix made of collagen and hyaluronic acid, and cells with nanofibre matrix made of dextran, pullulan and collagen;

FIG. 7 shows a photograph wrinkle reduction before and after the use of the nanofibre product of the present invention;

FIG. 8 illustrates a graph of the change of wrinkle with respect to time by using and without (control) using the nanofibre product of the present invention;

FIG. 9 illustrates a graph of the canthus elasticity variation after 28 days on one side of using nanofibre products and another side without (control) using nanofibre products of the present invention;

FIG. 10 shows a photograph of an exemplary coloured nanofibre for lip mask;

FIG. 11 illustrates exemplary assorted shapes of nanofibre mask;

FIG. 12 shows a table of absorbance values for lactate dehydrogenase assay after incubation with fibroblast and keratinocytes; and

FIG. 13 shows a table of microbial content at beginning, after stored at room temperature and 40° C. for 2 months.

### DETAILED DESCRIPTION

As noted above, described herein is polysaccharide and/or protein nanofibre matrix with functional ingredients for skin care and cosmetic products. More specifically, a biomaterial-based nanofibre matrix manufactured from a combination of polysaccharides and/or proteins for use in skin care and cosmetic products and manufactured by an electrospinning method. For advanced skin benefits, functional ingredients may be optionally incorporated in the nanofibre matrix during electrospinning and delivered into the skin with the nanofibres.

For the purposes of this specification, the term ‘about’ or ‘approximately’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘substantially’ or grammatical variations thereof refers to at least about 50%, for example 75%, 85% 95% or 98%.

The term ‘comprise’ and grammatical variations thereof shall have an inclusive meaning—i.e. that it will betaken to mean an inclusion of not only the listed components it directly references, but also other non-specified components or elements.

In a first aspect there is provided a nanofibre matrix comprising:

at least one type of polysaccharide; and/or optionally

at least one type of protein

the nanofibre matrix optionally carries with functional active(s) or ingredient(s) for advanced skin care application, wherein the nanofibres are produced from a mixed polysaccharide and/or protein solution with or without the added functional active(s) or ingredient(s) and electrospun into a dry nanofibre non-woven textile such as a dry nanofibre sheet; preferably, the dry nanofibre sheet is processed into different shapes and applied to different areas of skin; and wherein, preferably upon contact with wet skin, the nanofibres are dissolved rapidly or gradually dissolved into the skin for advanced therapeutic benefits.

The nanofibre matrix may be produced by electrospinning of 100% naturally derived polymers, preferably polysaccharides, proteins and/or the combinations thereof. The polysaccharides are preferably selected from, but not limited to any one of the following: dextran, pullulan; fucoidan; alginate; chitosan, chitin, amylose, amylopectin, pectin, hyaluronic add, plant-based polysaccharides in general; and/or combinations thereof.

The proteins may be selected, but not limited to any one or the following: whole chain collagen, denatured collagen, denatured whole chain collagen, hydrolysed collagen, collagen peptides, gelatin, keratin, hydrolysed keratin, zein, whey protein, hydrolysed whey protein, wheat protein, hydrolysed wheat protein, soy protein, hydrolysed soy protein, pea protein, hydrolysed pea protein, hemp protein, hydrolysed hemp protein and/or combinations thereof.

The present invention may comprise at least three categories of functional ingredients for:
I. Providing skin benefits for various skin conditions;
II. Providing colouring effect used for make-up products; and/or
Ill. Providing scent or flavour function for cosmetic products.

In one embodiment, the functional ingredients may include vitamins, ketones, fatty adds, terpenes, polyphenols, carotenoids, herb extracts and fruit extracts which may provide skin benefits which are targeted for various skin conditions. Examples of fruit extract functional ingredients may include grape seed extract, kiwifruit skin extract, tomato extract, bilberry extract and goji berry extract. Examples of plant and herbal extract functional ingredients may include aloe vera extract, mint extract, thyme extract, cannabinoid extract, elderflower extract, sage extract and rosemary extract. These functional ingredients can be incorporated within nanofibres during electrospinning to provide superior skin benefits, such as: antioxidant, wrinkle reduction, dark under-eye circle reduction, brightening, antibacterial, antimicrobial, acne treatment and moisturizing and the like. In this way, the use of nanofibre product is convenient and straightforward.

In some embodiments, functional ingredients may offer a colouring function by using the natural dye extracted from fruits or plants. Natural dye may be added into the nanofibre matrix at various concentrations to make coloured nanofibres that are suitable for us as natural makeup products. In this way, natural polymers such as polysaccharides or proteins have been demonstrated to have excellent abilities of carrying the natural dye.

It is envisaged that natural dyes may include, but should not be seen as limited to any one of the following: beetroot extract, elderberry extract, annatto extract, curcumin, astaxanthin, carrot root extract and/or combinations thereof. One or more combination of natural dye may be added into the matrix during electrospinning to make coloured nanofibres for makeup application. The natural dyes may be mixed homogenously in the solution, and evenly dispersed in the nanofibre matrix. Nanofibres may be dyed into a pink colour, orange colour, brown colour, dark colour and so on, depending on the selection of dye. Pink nanofibres may be used as lip product, or blush product. Brown or dark coloured nanofibres may be used as eyeshadow, eyebrow, contouring, hair dye product.

Functional ingredients may also be essential oils to make scented and flavoured nanofibre products. It is envisaged that essential oils may include, but should not be seen as limited to jojoba oil, peppermint oil, rosehip oil, sandalwood oil, lavender oil, squalane oil, squalene oil, manuka oil, tea tree oil, cannabinoid oil, coconut oil, olive oil, flaxseed oil, cinnamon oil, acai oil, argan oil, castor oil and/or combinations thereof. For example, peppermint oil can create a pleasant taste to nanofibre mask applied to the lips, or may act as a cooling effect to nanofibre applied to the skin.

Material Descriptions

As above, this invention uses natural polymers which are preferably polysaccharide and/or protein materials as a nanofibre matrix for skin care use. As will be appreciated by those skilled in the art, polysaccharides are polymeric carbohydrate molecules and made of long chains of monosaccharide units linked together by glycosidic bonds.

Preferably, the polysaccharide polymers may be selected from plant-based polysaccharides or animal-based polysaccharides. Animal-based polysaccharides may include, but are not limited to any one of the following: chitin, chitosan and hyaluronic acid. Plant-based polysaccharides may include, but are not limited to any one of the following: dextran, pullulan, fucoidan, pectin, hyaluronic add, alginate, amylose and amylopectin.

In preferred embodiments, the polysaccharide may be Hyaluronic acid. Hyaluronic add is composed of repeating D-glucuronic add and N-acetyl-D-glucosamine linked by a glucuronide bond. Hyaluronic acid is a naturally occurring polysaccharide and it is found in the extracellular matrix of all animal tissues. The biological functions of hyaluronic acid include: hydration, joint lubrication, space filling, and frameworks for cell migration. In this way, Hyaluronic acid has excellent water retention capacity (it can hold water up to 1000 times of its molecular weight), so has a very good moisturizing ability. In the aged skin, the synthesis speed of hyaluronic add is slower than the degradation speed of hyaluronic add, resulting in a dry, dull and wrinkled skin condition. It is important to supplement hyaluronic add in a daily routine to help reduce the transdermal water loss and reducing the depth of wrinkles. Due to the importance of hyaluronic add in skin, it is a very popular ingredient in the cosmetic market, offering the benefits of anti-ageing, moisturizing, wound healing and increasing elasticity. It is a naturally occurring polymer, so it is generally considered safe without causing irritation or allergy for most people. Most hyaluronic acid used in the market is plant-based and produced by microbial fermentation.

The molecular weight of hyaluronic add varies from a few thousands Dalton to million Daltons. High molecular weight of hyaluronic acid offers better moisturizing and plumping effect comparing to low molecular weight. However, high molecular weight hyaluronic acid has very poor skin penetration properties. Topical application of high molecular weight hyaluronic acid usually on the surface as a film and does not penetrate into the dermis layer. The most effective way to apply high molecular weight hyaluronic add for wrinkle reduction is by using fine needle to inject hyaluronic add directly into dermis layer. Needle injection needs to be carried out at spa and dermatology clinics and have risk of causing inflammation and swelling. Nanofibre matrix containing hyaluronic add overcomes these problems. In nano state (300 nm fibre dimension) any bulk macromolecules dissolution power over power its own molecular weight. Therefore, hyaluronic acid which is highly polar and hydrophilic breakdown smaller than size of skin pores (30 μm) in water much faster than its bulk state and penetrates the skin rapidly.

The proteins may be selected from plant-based protein, animal-based protein and/or the combination of both. Plant-based proteins may include, but are not limited to any one of the following: wheat protein, hydrolysed wheat protein, zein, soy protein, hydrolysed soy protein, pea protein, hydrolysed pea protein, hemp protein and hydrolysed hemp protein. Animal-based proteins may include, but are not limited to any one of the following: collagen, hydrolysed collagen, gelatin, keratin and hydrolysed keratin.

In this invention, preferably selecting collagen, hyaluronic add, dextran, pullulan and combinations thereof, to make nanofibre matrix.

To manufacture nanofibres, the polysaccharide and protein materials maybe dissolved in acidic solution and electrospun onto a supporting substrate. The acidic solution is preferably an aqueous acetic add. An example of polysaccharide and protein nanofibre matrix may be a combination of dextran, pullulan and hydrolysed collagen.

In yet further preferred embodiments, the polysaccharide polymer may be dextran. The dextran may be a water-soluble polymer and odourless. As is known in the art, it is produced by fermentation of sucrose where sucrose is from vegetables. Dextran is available in different molecular weight at 40 kDa, 60 kDa, 70 kDa, 110 kDa, 250 kDa etc.

In this way, Dextran has very good biocompatibility and has been used in pharmaceutical use, ophthalmic use, food additives and cosmetics use. For use in cosmetic products, it offers the benefits of moisturizing, stabilizer, soothing and firming the skin. Dextran in this invention has been demonstrated to be electrospun by itself or with other natural polymers. A nanofibre matrix containing dextran can offer the benefits of fast dissolution, fast absorption and moisturizing effect.

Pullulan is a water-soluble polymer and produced by fermentation of hydrolysed starch by the fungus Aureobasidium pullulans. Pullulan has been used for food additives, drug delivery and skin care products. Due to it is edible, tasteless, odourless properties, it is a good candidate to replace gelatin as a vegan alternative while behave similarly like gelatin. For example, Capsugel® from the U.S. released a product Plantcaps™ capsule made of pullulan to replace gelatin capsules for vegan consumers. It can provide all the benefits of gelatin capsules and provide elegant crystal-clear transparency look. Pullulan has been used as the oral refresher, for example, Listerine PocketPaks® oral care strips. When it is used for skin care products, it offers benefits of creating smoothness to products, film forming and instant skin-tightening effect. Due to it is edible nature, it is considered safe to use in cosmetic products. A nanofibre matrix containing pullulan can offer the benefits of fast dissolution, fast absorption, smoothing and skin tightening effect.

Fucoidan is a sulphated polysaccharide which contains a large portion of L-fucose and sulphate. It is a water-soluble polymer and extracted from brown seaweed. The methods used for extracting fucoidan include hot water extraction, acid extraction using hydrochloric acid or sulphuric acid, and salt extraction using calcium chloride. It is generally considered that good quality fucoidan has high L-fucose, high degree of sulphation, and low levels of contamination. It has been used as a food supplement, cosmetic product and pharmaceutical products. Fucoidan provides the benefits of increasing skin elasticity, UV protection, anti-ageing, antioxidant, anti-tumour, anti-Inflammatory and soothing effects.

Collagen is one of the main proteins present in animals. Collagen is made of amino acids and forms a triple helix structure. Intact triple helix collagen is the insoluble fibrous protein found in the extracellular matrix of the skin. The fibroblast is the most common cell that creates collagen. Compared to young skin, fibroblasts in aged skin synthesize lower levels of collagen. Therefore, supplement of collagen is necessary for aged skin, either by oral intake or topical application. Hydrolysed collagen is a hydrolysed form of collagen. As it is digestible, water soluble and easily absorbed, it has been widely used in various products for anti-ageing effect. Hydrolysed collagen can provide free amino acids to build blocks for the formation of collagen fibres and also acting as ligands, binding to receptors present on the fibroblast membrane and stimulate the production of new collagen.

For advanced skin benefits, functional ingredients may be incorporated into the nanofibre matrix.

It has been unexpectedly found by the inventors that due to the large surface area, nanofibres have been demonstrated to be a best platform for carrying functional ingredients. The high loading rate of functional ingredients may be achieved by using electrospinning methods.

The total weight of functional ingredients in the polymer matrix may be preferably between 0.1 wt. %-30 wt. %. It is envisaged that one functional ingredient or multiple functional ingredients may be incorporated within the matrix.

In preferred embodiments, naturally sourced actives/ingredients may be utilised for functional ingredients. This may include, but should not be seen as limited to vitamins, herbal extracts, fruit extracts, bio-based oil and vitamins.

Preferably, vitamins may include, but should not be seen as limited to: Vitamin A, Vitamin C, Vitamin D, Vitamin E, Vitamin K, Vitamin B1, B2 B3, B5 and/or their derivatives.

The fruit extracts described include, but should not be seen as limited to grape seed extract, kiwifruit skin extract, tomato extract, bilberry extract, goji berry extract.

The herbal extracts described include, but should not be seen as limited to aloe vera extract, thyme extract, elderflower extract, sage extract, rosemary extract and cannabinoid extract.

Bio-based oils described include, but should not be seen as limited to jojoba oil, peppermint oil, lavender oil, squalane oil, squalene oil, tea tree oil, coconut oil, olive oil, flaxseed oil, cinnamon oil, acai oil, argan oil, castor oil and cannabinoid oil.

It is envisaged to manufacture nanofibre matrix containing functional ingredients, the functional ingredients may be added into premixed polysaccharide/protein solution. In alternative embodiments, the polysaccharide and protein may be added into the premixed functional ingredient solution.

The solvent may be an aqueous acidic solvent where the viscosity and conductivity of solution may be changed with added functional ingredients.

The polymer solution may be formulated into dry nanofibre sheets at high voltages, wherein the functional ingredients may be successfully incorporated into the nanofibre matrix by electrospinning.

An example of utilising vitamins in a facial mask may be to incorporate Vitamin C derivatives in the nanofibre matrix. Aside from the benefits from polysaccharide polymers, the Vitamin C may offer other benefits such as antioxidant, stimulating collagen synthesis and inhibiting melanogenesis. In other embodiments, Rosemary herb extract may be incorporated in the nanofibre matrix, offering antioxidant properties too. Tea tree oil may be incorporated into the nanofibre matrix and may offer the skin benefits of antibacterial and may be useful for acne treatment.

It is envisaged that the dry nanofibre sheet may be processed into assorted shapes, like whole facial masks, under-eye patches, nose masks, forehead masks, chin masks, cheek masks, neck masks and other shapes and configurations to target different areas of skin.

A full beauty treatment may include a set of whole facial masks, under-eye patches, nose mask, forehead mask, chin mask, cheek mask and neck mask where each type of mask may have a different functional ingredient. For example, if a user has a skin problem described as below: prone to have acne around chin, cheek and nose areas, wrinkles around forehead and neck, dark circle around eyes and general dry skin. A beauty package may include a whole facial mask which is made of nanofibre matrix with functional ingredient which provide general moisturizing effect, like aloe vera extract. Under eye patches may be made of nanofibre matrix with Vitamin C which can offer the dark circle reduction effect. Chin mask, cheek mask, T-zone and nose mask can be made of nanofibre matrix with tea tree oil for antibacterial and acne treatment. Forehead mask and neck mask may be made of nanofibre matrix with Vitamin A or derivatives for anti-wrinkle reduction. Each type of mask only requires to be left on the targeted area for a few seconds. Nanofibres carrying with functional ingredients are efficiently delivered to the skin and offer different functions and benefits to different skin area. This dry nanofibre sheet mask is an innovative technology which overcomes problems associated with traditional sheet masks.

The purposes of the functional ingredients are not limited to its skin benefit effects like antioxidant, brightening, moisturizing, wrinkle reduction, but it can also provide other properties, like colouring. The functional ingredients may be natural dye extracts. In this way, the nanofibre matrix with natural dye may be used for lipstick or a lip treatment. It is also envisaged that the nanofibre matrix may be manufactured from edible ingredients. In this way, it will be safe to use as lipstick or a lip treatment.

The nanofibres containing hyaluronic acid and peppermint oil also have shown very good plumping effects.

In a second aspect there is provided a method of manufacturing a nanofibre matrix as substantially described above.

The method to manufacture a polysaccharide/and or protein nanofibre matrix may optionally contain functional actives or ingredients for skin care products.

The polysaccharide/and or protein nanofibre matrix may be produced by electrospinning methods that result in water-soluble polysaccharide polymers becoming dry nanofibres. The dry polysaccharide nanofibre matrix with or without containing functional ingredients may immediately and efficiently be absorbed by the wet skin and provide superior therapeutic benefits.

In one embodiment, the polysaccharide polymers may be water soluble polymers.

The proteins may can include, but should not be seen as limited to collagen, hydrolysed collagen, gelatin, whey protein, hydrolysed whey protein, keratin, hydrolysed keratin, wheat protein, hydrolysed wheat protein, soy protein, hydrolysed soy protein, pea protein, hydrolysed pea protein, hemp protein, and/or hydrolysed hemp protein.

The nanofibre matrix may be made of at least one type of polysaccharide and/or at least one type of protein. One or more types of polysaccharides and/or one or more types of proteins may be selected for producing nanofibre matrix.

Electrospinning Solution

The method to produce polysaccharide nanofibre matrix may dissolve polysaccharide and protein in aqueous solvents. By using electrospinning methods, the polysaccharide and protein solution turn into nanofibres under high voltages, wherein the nanofibres may be deposited on the collecting substrate materials.

As above, the nanofibre matrix may be produced by electrospinning methods. The first step is to make an electrospinning solution. The polysaccharides and/or proteins are added in aqueous solution and mixed for 1-3 hours until all materials are fully dissolved. Here the aqueous solution is preferably 10-30 wt. % ethanol or acetic acid in water. The total polymer concentration in solution may be between 10 wt. %-50 wt. % depending on the selected composition of polymers, and molecular weight of polymers. The viscosity of solution may be between 100 cst-400 cst and the conductivity of solution may be between 1-5 mS/cm. In this invention, the polymers are natural polymers and the prepared electrospinning solution does not contain any synthetic polymers, preservatives, crosslinking agents and surfactants.

Electrospinning Conditions

As above, to prepare electrospinning solution, the polysaccharide and protein materials may be added sequentially into solvents under stirring for a few hours. In alternative embodiments, the polysaccharide solution and protein solution may be prepared separately and then the two solutions mixed together.

The electrospinning method may include a needle-less electrospinning method.

The electrospinning parameters, especially voltages, current, electrospinning distance, solution feed rate, need to be adjusted depending on the viscosity and conductivity of the solution, ultimately determined by the selection of polymers and molecular weight of polymers. Ambient environment conditions such as humidity and temperature are critical in electrospinning process. The preferable environment relative humidity in this invention is between 25%-50% and temperature may be between 15° C.-35° C. The prepared electrospinning solution is added into electrospinning apparatus. Under the high voltages, polymers are electrospun into nanofibres. The solvents are evaporated during the electrospinning process, so the produced nanofibres are the dry sheet without containing solvents. The produced nanofibre matrix is protein nanofibres, polysaccharides nanofibres or the combination of protein and polysaccharide nanofibres.

The produced nanofibre matrix combination of polysaccharides and proteins may provide both benefits from polysaccharides and proteins to skin.

The functional ingredients may be incorporated within the nanofibre matrix to provide more advanced skin benefits. It has been found that Nanofibres are good stabilizers, carriers and delivery platforms for functional ingredients.

The diameter of nanofibre may be between 10 nm to 2 microns.

The density of nanofibre matrix may be between 1 gsm-100 gsm.

The weight ratio of functional ingredients in the nanofibre matrix may be between 0.1 wt %-30 wt %.

The embodiments described above may also be said broadly to consist in the parts, elements and features referred to or indicated in the specification of the application, individually or collectively, and any or all combinations of any two or more said parts, elements or features.

Nanofibre Matrix with Functional Ingredients

To achieve advanced skin benefits, functional ingredients are incorporated into the nanofibre matrix. Due to their large surface area, nanofibres have been demonstrated to be the best platform for carrying functional ingredients. High loading rates of functional ingredients can be achieved by using the electrospinning method. The total weight of functional ingredients in a polymer matrix is preferably between 0.01 wt. %-30 wt. %. One functional ingredient or multiple functional ingredients can be incorporated within a matrix. Functional ingredients can be divided into three categories:

I. Providing skin benefits for various skin conditions;

II. Providing colouring effect for makeup products; and

III. Providing scent or flavour function for cosmetic products.

A nanofibre matrix containing functional ingredients is produced by the following steps. Firstly, polysaccharides and/or proteins are dissolved in an aqueous solution. Here the aqueous solution is 10 wt. %-30 wt. % of ethanol or acetic acid in water. The functional ingredients are then added into polysaccharide/protein solution and mixed well until the functional ingredients are dissolved or uniformly dispersed in the polymer solution. The polymer solution containing functional ingredients are added into electrospinning apparatus. After electrospinning, the functional ingredients are successfully incorporated within the nanofibre matrix.

Functional ingredients which offers skin benefits can be selected from, but not limited to vitamins, ketones, fatty acids, terpenes, polyphenols, carotenoids, plant extracts, herbal extracts, fruit extracts, plant extract, essential oils and combinations thereof. The vitamins may include, but not limited to: Vitamin A, Vitamin C, Vitamin D, Vitamin E, Vitamin K, Vitamin B1, B2, B3, B5 or their derivatives. Fruit extracts include, but are not limited to: grape seed extract, kiwifruit skin extract, tomato extract, bilberry extract and goji berry extract. Plant/herbal extracts include but not limited to aloe vera extract, thyme extract, elderflower extract, cannabinoid extract, sage extract and rosemary extract. For example, a nanofibre matrix containing the rosemary herb extract offers the antioxidant properties. A nanofibre matrix containing the Vitamin C offers the skin brightening effect. A nanofibre containing Vitamin A offers the acne treatment function.

Functional ingredients for skin benefit effects such as: antioxidant, brightening, moisturizing, wrinkle reduction, but it can also provide other functionality like colouring. Coloured nanofibres can be used for makeup products. To make coloured nanofibres, natural dye is added into the polymer solution before electrospinning. Natural dye includes but not limited to beetroot extract, elderberry extract, annatto extract, curcumin, astaxanthin, carrot root extract and combinations thereof. Beetroot extract shows red/pink colour due to it contains the pigment betanin. The main pigment in elderberry extract is cyanidin 3-glucoside. The main pigment in annatto extract is bixin and norbixin which are present as yellow to orange colour. Beta-carotene in carrot root extract contributes to the orange colour. The nanofibre matrix with natural dye can be used for nail polish, lip, blush, eyebrow product, eyeshadow, contouring and hair products. As noted above, the nanofiber matrix in this invention includes all natural ingredients. Therefore, it is safe to use, and is especially beneficial in lip type products.

Aside from skin benefits, colouring effect, functional ingredients can also be selected to provide scent to nanofibre products, such as adding essential oils into nanofibres. Essential oils include but are not limited to jojoba oil, peppermint oil, lavender oil, squalane oil, squalene oil, tea tree oil, coconut oil, olive oil, manuka oil, cannabinoid oil, flaxseed oil, cinnamon oil, acai oil, argan oil, castor oil and combinations thereof.

Further, where specific integers are mentioned herein which have known equivalents in the art to which the embodiments relate, such known equivalents are deemed to be incorporated herein as if individually set forth.

### Working Examples

The above described products, compositions, methods and uses thereof are now described by reference to specific examples.

### Example 1

Morphology of Nanofibre Matrix

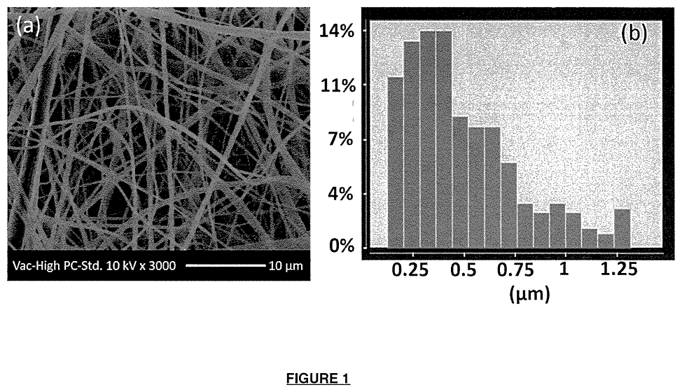

In the present invention, the morphology of produced nanofibre matrix is analysed by scanning electron microscopy (SEM) and fibre diameter distribution is analysed by Fibraquant software as is shown in FIG. 1.

As is shown, the polymer solution containing dextran, pullulan and collagen has been successfully electrospun into nanofibres. In particular, the nanofibre matrix is a continuous network as shown in FIG. 1(a) and fibre diameter distribution is shown in FIG. 1(b). The average diameter of this type of matrix is 495 nm and the median diameter is 426 nm. It is envisaged that the embodiments of this invention covers fibre diameters between 10-2000 nm.

### Example 2

Application Method

To use a nanofibre matrix mask, the skin is first wet with clean water. The water can be ice water, room temperature water or warm water which provide different options to consumers. The water also can be replaced by aqueous toner or mist or an aerosol.

The nanofibre matrix is applied directly onto the wet skin and nanofibres will be instantaneously absorbed by skin. There is no rinsing off required.

### Example 3

Nanofibre Matrix Skin Penetration Properties

The cosmetic products manufactured by the nanofibre matrix are absorbed by the skin and penetrate deeper into the skin over time. Optical coherence tomography is used as an analytical tool to demonstrate this functionality. As is known in the art, optical coherence tomography is a non-invasive method that has been widely used in dermatology for skin layer structure study, disease diagnostics and drug delivery.

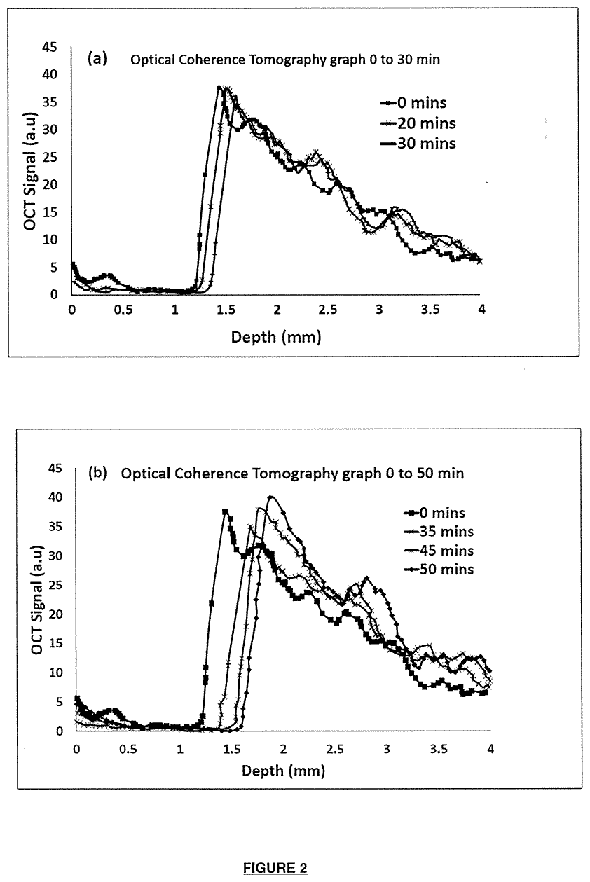

For experimentation, the nanofibre matrix is applied onto a stillborn pig skin and scan the cross section of skin at t=0, 20 mins, 30 mins, 35 mins, 45 mins and 50 mins. The optical signal at each time point is shown in FIG. 2. Here the nanofibre matrix comprises dextran, pullulan and collagen, without other ingredients.

At t=0 when the nanofibres are just applied onto the surface of skin, the peak is positioned at the depth of 1.44 mm. At t=20 mins, the peak is shifted to the depth of 1.53 mm. This indicates that the penetration of nanofibres in the skin layer occurs within 20 mins. The peak continues to shift to the depth of 1.6 mm after 30 mins, 1.7 mm after 35 mins, 1.76 mm after 45 mins and 1.89 after 50 mins. This indicates that this nanofibre matrix and ingredients keeps penetrating into the skin and penetrates further into the skin over time.

### Example 4

Plant Based or Vegan Nanofibre Matrix

The vegan products disclosed in this invention are achieved by selecting the plant-based polysaccharides and/or plant-based proteins. Vegan products have become increasingly popular as consumers consider animal cruelty, sustainability, and the environmental footprint of farming associated with animal-based products.

The nanofibre matrix can be produced by using 100% plant-based polymers. In this example, the vegan nanofibre matrix is produced by using dextran, pullulan and fucoidan. The morphology of vegan nanofibre matrix and fibre diameter distribution are shown in FIG. 3. To manufacture this nanofibre matrix, a polymer solution is firstly prepared by adding dextran, pullulan and fucoidan into an aqueous acetic acid solution, and stirred for 1-3 hours until all polymers are fully dissolved. The added weight of fucoidan in polymer is 100 mg/g. Then the polymer solution is added into the electrospinning apparatus and electrospun into nanofibre matrix under high voltage.

The vegan nanofibre matrix is a continuous network of fibres. The average diameter of the nanofibres is 320 nm and the median diameter is 275 nm. Dextran, pullulan and fucoidan can offer different cosmetic benefits. Dextran has superior moisturizing benefits, pullulan has excellent skin tightening effects, and fucoidan can offer benefits such as antioxidant, anti-inflammatory and UV protection properties. This vegan nanofibre matrix offers multiple cosmetic benefits, while it is very safe to use. Moreover, they are all edible materials and have been added as supplements in the food industry. If food grade dextran, pullulan and fucoidan are utilised in producing the nanofibres, the resulted nanofibre matrix can be considered as an “edible” cosmetic product.

To prove the existence of fucoidan in the vegan nanofibre matrix, spectrophotometry method is used in known fashion. Fucoidan contains a large proposition of L-fucose, so L-fucose can be used as a marker or indicator of the presence of fucoidan in the matrix. The produced vegan nanofibre matrix is dissolved in Milli-Q water at an approximate concentration of 100 mg/mL An aliquot of the solution is diluted in 12M sulfuric acid and heated at 100° C. for 15 minutes while constant stirring. Following cooling to room temperature, 3% w/v L-cysteine hydrochloride is added. A calibration curve is generated by preparing a solution of L-fucose in the same manner. A purified extract of fucoidan is analysed as a positive control for the experiment. Samples and standards are aliquoted into a microplate and read using a microplate reader at 405 nm. The vegan nanofibres are analysed in triplicates and the average value of L-fucose in nanofibres is 147 mg/g. As dextran and pullulan do not contain L-fucose, the detected L-fucose level is from fucoidan. This demonstrates that the presence of fucoidan in the nanofibre matrix. The level of L-fucose content is highly dependent on the extraction method and seaweed species. The added fucoidan in total polymer weight is 100 mg/g. This means that the fucoidan utilised to manufacture the vegan nanofibre matrix contains a high level of L-fucose.

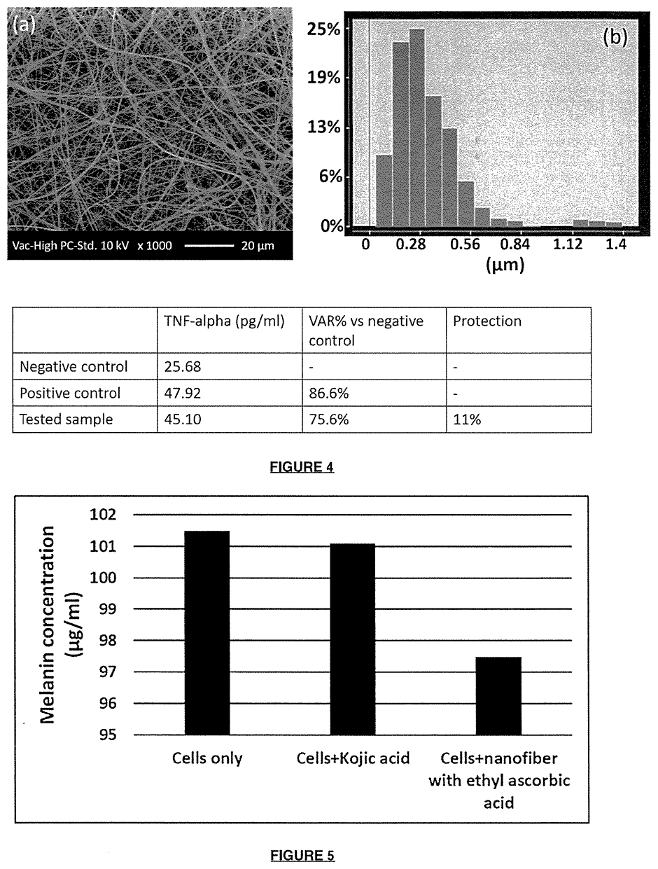

The vegan nanofibre matrix comprising dextran, pullulan and fucoidan shows a good anti-Inflammatory property and it is demonstrated by an in vitro study. A human reconstructed epidermis is put in contact with the vegan nanofibres (topical application to mimic the real condition of use) and treated with an irritant inflammatory agent such as sodium dodecyl sulfate (SDS) to assess the capability of vegan nanofibers to counteract the inflammatory event. The evaluation is performed by using a non-treated epidermis as negative control and the epidermis treated only with the inflammatory agent (SDS) as a positive control. TNF-alpha is often associated with the inflammatory process and oxidative stress as an acute pro-inflammatory marker. After 24 hours incubation, the concentrations of proinflammatory cytokine TNF-alpha in the tested sample and control samples are obtained as shown in FIG. 4, giving the indication on the inflammatory status of the epidermis.

The concentration of TNF-alpha is 25.68 μg/ml in non-treated epidermis, 47.92 μg/ml in epidermis treated with 0.1% inflammatory agent SDS, 45.1 μg/ml in epidermis treated with vegan nanofibres and 0.1% inflammatory agent. The epidermis treated with vegan nanofibres decreases the TNF-alpha release. These results therefore demonstrate that the vegan nanofibre produced by dextran, pullulan and fucoidan has anti-Inflammatory property and protection ability against inflammatory agents.

### Example 5

Formulation Development: Brightening

A further example of the present invention is a nanofibre matrix containing a Vitamin C (ascorbic acid) derivative for skin brightening effect. In this example, the nanofibre matrix is produced by collagen and hyaluronic add. Ethyl ascorbic acid is a stable form of Vitamin C, and is added as a functional ingredient incorporated within nanofibre matrix. To manufacture this formulation for brightening cosmetic products, firstly the electrospinning solution is prepared. Collagen and hyaluronic acid are added in aqueous acetic acid solution and mixed well until they are fully dissolved. Ethyl ascorbic add is then added into the polymer solution and mixed well until it is fully dissolved. The concentration of ethyl ascorbic acid in polymer in this invention is preferably between 0.05%-10%. The solution is added into the electrospinning apparatus. Electrospinning parameters and ambient environment conditions are adjusted to optimize the production of this formulation. The produced nanofibre matrix contains ethyl ascorbic acid. The brightening effect of this formulation is assessed by in vitro study.

In vitro study is used to evaluate the brightening effect and is determined by analysing the melanin production of melanocytes. The level of melanin produced by melanocytes determines the colour of skin, therefore, the brightening effect can be evaluated by the impact of formulation on the melanin production of melanocytes. In this study, human epidermal melanocytes are utilised. Kojic acid, traditionally used as a brightening ingredient, is used as the positive control. Kojic add is prepared in 15% ethanol/Hanks balanced salt solution and added into cell culture to give final concentration of 42 μg/ml in cell culture. 0.8 cm circles of nanofibre is directly applied into the cell culture to give a final concentration of ethyl ascorbic add of 3 μg/ml in cell culture. FIG. 5 presents the result of melanin concentration in cells only, cells treated with kojic acid and cells treated with nanofibres after 96 hours incubation time.

The absorbance of each sample is read at 405 nm and then compared with a standard curve of synthetic melanin to estimate the concentration of melanin in the cell culture. The displayed results are the average value of triplicates. The melanin concentration is 101.5 μg/ml in cells only, 101.1 μg/ml in cells with kojic add and 97.48 μg/ml in cells with nanofibres. Kojic acid shows slight inhibition to melanin production, but is not statistically significant. This is probably due to the added kojic add concentration being very low. The Nanofibre matrix with ethyl ascorbic acid shows 4% inhibition to melanin production comparing to the cells only. The concentration of ethyl ascorbic add in cell culture is only 3 μg/ml, 14 times lower than the concentration of kojic add. However, the melanin inhibition ability is better than kojic acid. This indicates that the nanofibre matrix is a good platform to carry active ingredients and efficiently maximize the benefits of functional ingredients. This formulation can be used for whole face masks aiming for a brightening effect, or under-eye patches for reducing the dark under-eye circles.

### Example 6

Formulation Development: Anti-Ageing and Wrinkle Reduction

In Vitro Evaluation: Elastin Stimulation

By selecting different types of polysaccharides and proteins, the nanofibre matrix can realise different purposes for skin benefits. An example of developed nanofibre matrix in this present invention shows enhanced elastin stimulation properties from in vitro studies. This nanofibre matrix can be used to reduce the wrinkles and improve the elasticity of skin. FIG. 6 shows the elastin concentration of cells only, cells with nanofibre matrix comprising collagen and hyaluronic add, as well as cells with a nanofibre matrix comprising dextran, pullulan and collagen, after 24 hours incubation in skin fibroblast.

After 24 hours incubation in skin fibroblast culture, the concentration of produced elastin is 0.143 ng/ml in cells only, 0.544 ng/ml in the nanofibre matrix comprising collagen and hyaluronic acid, and 0.165 ng/ml in the nanofibre matrix comprising dextran, pullulan and collagen. Both formulations of nanofibre matrix demonstrate enhanced elastin stimulation abilities. The collagen and hyaluronic add nanofibre matrix stimulate the elastin production by 280% comparing to cells only. The dextran, pullulan and collagen nanofibre matrix stimulate the elastin production by 15% comparing to cells only. Although both nanofibre matrix show elastin stimulation properties, due to nanofibre matrix made of collagen and hyaluronic add shows much better performance, this matrix will be preferably selected for anti-wrinkle properties to reduce the signs of ageing.

In Vivo Evaluation: Wrinkle Reduction.

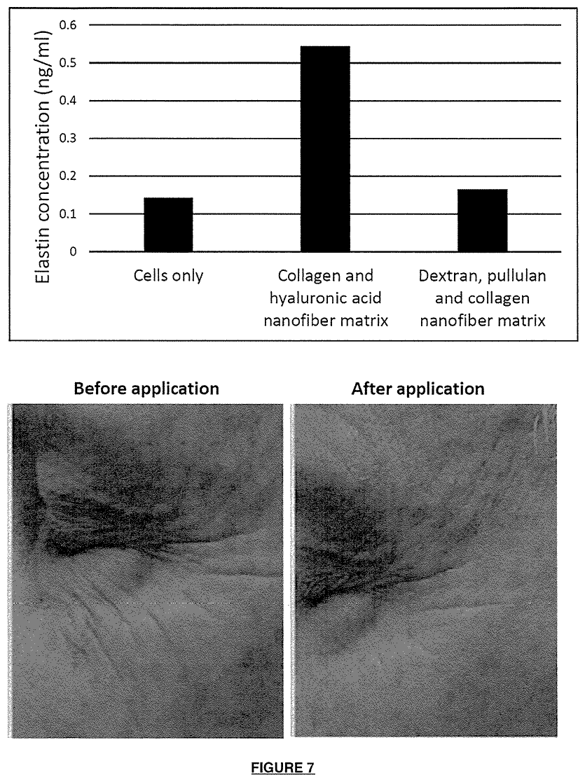

The functional ingredients can be incorporated into the nanofibre matrix to provide additional benefits. The functional ingredients can be selected from fruit extracts. An example of a developed formulation comprising collagen and hyaluronic add nanofibre matrix with kiwifruit and grapeseed extracts has shown excellent wrinkle reduction effect—best seen in FIG. 7 showing before and after use of this nanofibre formulation.

A volunteer applies the nanofibre product around the corner of their eye and photos are taken before and after application. As can be seen, the quantity and depth of wrinkles are clearly reduced after using this nanofibre formulation.

Another independent in Vivo study has been conducted on a focus group of 30 middle aged volunteers (age distribution between 32 to 58, with an average age of 45) in order to demonstrate the anti-ageing properties of the collagen and hyaluronic add matrix formulation with kiwifruit and grapeseed extracts. The volunteers only applied the nanofibre product to one side of the under eye area, and left the other side without applying the nanofibre formulation as a control. The volunteers used this nanofibre product once a day and consecutively use it for 28 days. The information of wrinkle and elasticity is obtained and analysed by VISIA skin analysis software in known fashion. The Information was collected at day 0, day 14 and day 28.

FIG. 8 represents the results of variation of wrinkles with time on one side of the face using the nanofibre product and the other side without using the nanofibre product.

Before the test started, at DO, the side selected to apply nanofibre product appears to have more serious wrinkles. The wrinkles gradually reduced with time after starting to use nanofibre products and wrinkles are reduced by 3% in 28 days. In control side, without using nanofibre product, the wrinkles gradually increased, and increased by 8% in 28 days.

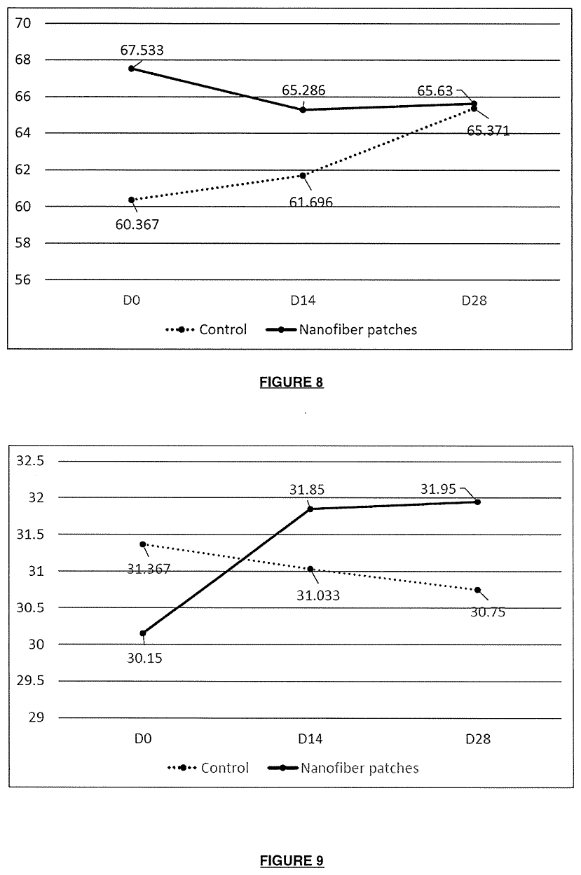

The same test protocol is used to test the change of canthus elasticity around eye areas and results are shown in FIG. 9.

As aforementioned, the volunteers only applied one side with the nanofibre product and did not apply anything on the other side as control. The starting point at DO shows the control side has better elasticity than tested side. However, without applying nanofibre products, the elasticity gradually reduces with time and it reduces by 2% after 28 days. The test side where volunteers apply nanofibre product once per day and use consecutively for 28 days, the elasticity increased by 6%.

Both in Vitro and in Vivo studies have demonstrated that the formulation of collagen and hyaluronic acid nanofibre matrix with kiwifruit and grapeseed extract have excellent wrinkle reduction qualities and can be used to reduce the signs of ageing when used as a cosmetic product.

### Example 7

Formulation Development: Coloured Nanofibre for a Lip Mask

A natural dye is selected as a functional ingredient to manufacture coloured nanofibres for makeup products. FIG. 10 shows an example of pink nanofibre in a lip shape which can be used for lip mask application. The pink nanofibre is realised by adding betanin (extracted from beetroot) in collagen and hyaluronic add polymer solution before electrospinning.

Natural polymers such as proteins and polysaccharides have demonstrated their excellent properties of carrying natural dye. To produce this formulation, firstly collagen and hyaluronic acid is mixed well in aqueous acidic add solution. Betanin is then added into polymer solution and mixed well with polymer solution. After electrospinning, betanin is successfully incorporated into the collagen and hyaluronic add nanofibre matrix. The whole nanofibre sheet is uniformly dyed with colour without seeing any aggregated particles. The pink nanofibres are achieved due to the added betanin. Unlike other lip products which contain synthetic products and preservatives. This lip product only contains three ingredients. Every ingredient is natural and has its own functionality. Collagen is beneficial for reducing the wrinkles, hyaluronic add has moisturizing and plumping effects while belanin provides colour. The coloured pigment remains on the surface of the lips as a uniform coating. As these three ingredients are edible, they are considered safe to use.

### Example 8

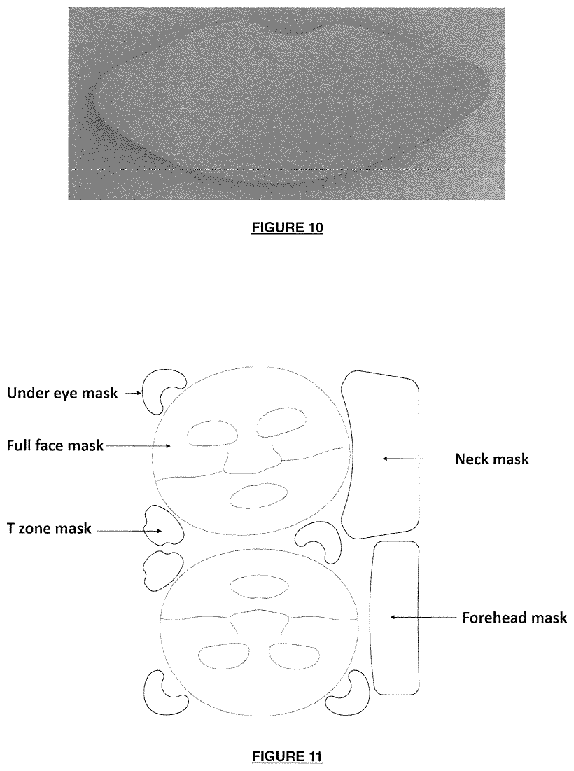

The manufactured dry nanofibre sheet can be processed into assorted shapes, such as a whole facial mask, an upper face mask, a lower face mask, under-eye patches, a nose mask, a forehead mask, a chin mask, a cheek mask, a neck mask and target different areas of skin, as shown in FIG. 11.

For example, a full beauty treatment regime can include a set of whole facial masks, under-eye patches, a nose mask, a forehead mask, a chin mask, a cheek mask and a neck mask where each type of mask might contain different functional ingredients.

Usually consumers have combinations of skin conditions. For example, a user has a skin problem described as below: prone to have acne around chin, cheek and nose areas, wrinkles around forehead and neck, dark circles around eyes and general dry skin. A customized beauty package can include a whole facial mask which is manufactured of a nanofibre matrix with functional ingredients that provide general moisturising effects, for example aloe vera extract. Under eye patches are manufactured of a nanofibre matrix with Vitamin C which can offer the dark circle reduction effect. A Chin mask, cheek mask and nose mask can be manufactured of nanofibre matrix with Vitamin A for acne treatment. A forehead mask and neck mask are manufactured of nanofibre matrix with kiwifruit and grapeseed extracts for anti-wrinkle reduction. Each type of mask only requires to be left on the targeted area only for a few seconds. Nanofibres carried with functional ingredients are efficiently absorbed by skin and offer different functions and benefits to various skin conditions. Hence, this dry nanofibre sheet mask is inventive which overcomes the problems with existing traditional sheet masks.

Furthermore, this invention also gives consumers options to manually cut the nanofibres into any shapes according to their needs. This invention can overcome the problems of the current uni-size mask which is not always a good fit for everyone and are not easy to cut into the shapes as desired. For example, for acne treatment, users can cut nanofibre products and only apply it to the infected area without impacting other parts of their skin.

### Example 9

Safety and Stability of Nanofibre Products

Cytotoxicity Test

The nanofibre products have been tested for cytotoxicity in vitro to evaluate its safety for use. The test is carried out on skin fibroblast and keratinocytes. The results are determined by the activity of lactate dehydrogenase. Three formulations are tested: collagen and hyaluronic acid nanofibre matrix, collagen and hyaluronic acid nanofibre matrix with kiwifruit and grapeseed extracts, as well as dextran, pullulan and collagen nanofibre matrix. FIG. 12 illustrates the level of lactate dehydrogenase from these different formulations in skin fibroblast and keratinocytes.

After 72 hours incubation time, none of the above formulations exhibited any toxic effect on the fibroblast and keratinocytes.

Microbiol Test

The disclosed invention of nanofibre matrix manufactured of polysaccharides and/or protein does not contain any preservatives. The microbial content results of nanofibre matrix demonstrates that it does not require preservatives.

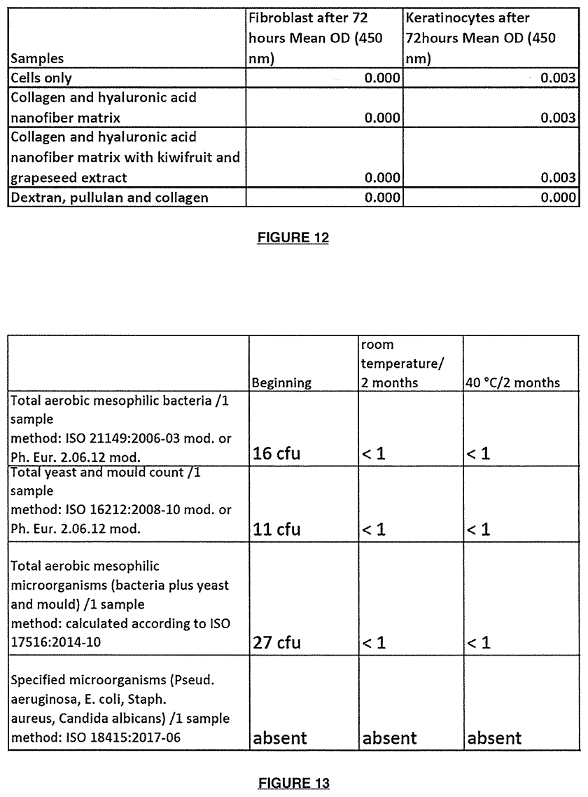

In particular, FIG. 13 shows the results of microbial content at beginning and after storing at room temperature and 40° C. after 2 months' time. This formulation is made of collagen and hyaluronic acid nanofibre matrix with kiwifruit and grapeseed extract.

The total aerobic mesophilic bacteria, total yeast and mould count, and total aerobic mesophilic microorganisms are all less than 1 cfu after being stored at room temperature and 40° C. after 2 months' time. In controlled conditions, storing at 40° C. can represent the results of storing at room temperature for 8 months. The specified microorganisms are absent. This demonstrates that the cosmetic products manufactured by this invention do not need preservatives

Aspects of the present invention have been described by way of example only and it should be appreciated that modifications and additions may be made thereto without departing from the scope of the claims herein.

What is claimed is:

1. A nanofibre matrix comprising of:
at least one type of polysaccharide; and/or optionally
at least one type of protein
the nanofibre matrix optionally carries with functional active(s) or ingredient(s) for advanced skin care application, wherein the nanofibres are produced from a mixed polysaccharide and/or protein solution with or without the added functional active(s) or ingredient(s) and electrospun into a dry nanofibre non-woven textile such as a dry nanofibre sheet; preferably, the dry nanofibre sheet is processed into different shapes and applied to different areas of skin; and
wherein, preferably upon contact with wet skin, the nanofibres are dissolved are gradually dissolved into the skin and release any actives or sits on the skin to release any actives for advanced therapeutic benefits.

2. The nanofibre matrix according to claim 1, wherein the nanofibre matrix is derived from natural polymers, wherein the matrix is solvent free, surfactant free, preservative free and crosslinker free; and free from synthetic or petroleum derived chemicals.

3. The nanofibre matrix according to claim 1 or claim 2, wherein the polysaccharides are animal-based or plant-based or the combination of both.

4. The nanofibre matrix according to any of the preceding claims, wherein the animal-based polysaccharides include chitin, chitosan, hyaluronic add or combinations thereof and the plant-based polysaccharides include dextran, pullulan, fucoidan, alginate, pectin, hyaluronic add, amylose, amylopectin or combinations thereof.

5. The nanofibre matrix according any of the preceding claims, wherein the proteins are animal-based or plant-based or the combination of both.

6. The nanofibre matrix according to any of the preceding claims, wherein the animal-based proteins include collagen, hydrolysed collagen, denatured collagen, denatured whole chain collagen, gelatin, keratin, hydrolysed keratin or combinations thereof and the plant-based proteins include whey protein, hydrolysed whey protein, zein, wheat protein, hydrolysed wheat protein, soy protein, hydrolysed soy protein, pea protein, hydrolysed pea protein, hemp protein, hydrolysed hemp protein or combinations thereof.

7. The nanofibre matrix according to any of the preceding claims, wherein the nanofibre matrix is made of at least one type of polysaccharide and/or at least one type of protein; and preferably, one or more types of polysaccharides, and/or one or more types proteins are selected for producing nanofibre matrix.

8. The nanofibre matrix according to any of the preceding claims, wherein the nanofibre matrix is a combination of polysaccharides and/or proteins and provide benefits from both polysaccharides and proteins to skin.

9. The nanofibre matrix according to any of the preceding claims, wherein the functional ingredients are incorporated within the nanofibre matrix to provide more advanced skin benefits; and preferably, the nanofibre matrix is a good stabilizer, carrier and delivery platform for functional ingredients.

10. The nanofibre matrix according to any of the preceding claims, wherein the skin benefits of the functional ingredients include but are not limited to moisturizing, brightening, wrinkle reduction, dark circle reduction, antibacterial, tightening, antioxidant and acne treatment.

11. The nanofibre matrix according to any of the preceding claims, wherein the functional ingredients offer other benefits of plumping, glowing and colouring.

12. The nanofibre matrix according to any of the preceding claims, wherein the functional ingredients include but are not limited to vitamins, fruit extracts, plant extracts, herbal extracts and bio-based or essential oils.

13. The nanofibre matrix according to claim 12, wherein the vitamins include but are not limited to Vitamin A, Vitamin C, Vitamin D, Vitamin E, Vitamin K, Vitamin B1, B2, B3, B5 or their derivatives or combinations thereof.

14. The nanofibre matrix according to claim 12, wherein the fruit extract functional ingredients include but are not limited to grape seed extract, kiwifruit skin extract, tomato extract, bilberry extract, goji berry extract.

15. The nanofibre matrix according to claim 12, wherein the plant or herbal extract functional ingredients include but are not limited to aloe vera extract, thyme extract, elderflower extract, sage extract, rosemary extract, cannabinoid extract.

16. The nanofibre matrix according to claim 12, wherein the essential oils offer a scented function and include but are not limited to jojoba oil, peppermint oil, lavender oil, rosehip oil, sandalwood oil, lavender oil, squalane oil, squalene oil, manuka oil, tea tree oil, cannabinoid oil, coconut oil, olive oil, flaxseed oil, cinnamon oil, acai oil, argan oil, castor oil and combinations thereof.

17. The nanofibre matrix according to any of the preceding claims, wherein a composition of functional ingredients is added into the polysaccharide and/or protein nanofibre matrix to obtain multiple advanced benefits; and preferably, the nanofibre matrix carrying with different functional ingredients provides different functions for skin.

18. The nanofibre matrix as claimed in claim 17, wherein the functional ingredient provides a colouring effect to the nanofibers, and wherein the coloured nanofibres are used for makeup products such as blush, eyeshadow, eyebrow product, nail polish, lip products, contouring and hair dye products.

19. The nanofibre matrix as claimed in claim 18, wherein the coloured nanofibres of the nanofibre matrix are derived from natural dye extracted from plants, vegetables or fruits, and wherein the natural dye includes but is not limited to beetroot extract, elderberry extract, annatto extract, curcumin, astaxanthin, carrot root extract and combinations thereof.

20. The nanofibre matrix according to any of the preceding claims, wherein the matrix can carry one type of functional ingredient or multiple types of functional ingredients.

21. The nanofibre matrix according to any of the preceding claims, wherein cosmetic products are applied onto the wet skin and nanofibres are absorbed by the skin to provide skin benefits, scent function, or colouring effects.

22. The nanofibre matrix according to any of the preceding claims, wherein skin benefits and dissolution rate of the nanofibre into the skin are varied by functional ingredients.

23. The nanofibre matrix according to any of the preceding claims, wherein the diameter of the nanofibre is between 10 nm to 2 microns.

24. The nanofibre matrix according to any of the preceding claims, wherein the density of the nanofibre matrix is between 1 gsm-100 gsm.

25. The nanofibre matrix according to any of the preceding claims, wherein the weight ratio of functional ingredients in the nanofibre matrix is between 0.1 wt. %-30 wt. %.

26. A dry nanofibre sheet made of the nanofibre matrix according to any of claims 1-25, wherein the dry nanofibre sheet is be used to be processed into different shapes for using in different areas of skin: whole face, upper face mask, lower face mask, under eyes, forehead, nose, chin, cheek, lip, T-zone and neck.

27. The dry nanofibre sheet according to claim 26, wherein the dry nanofibre sheet is manually cut by users to any desired shape to fit into their needs.

28. A cosmetic product in the form of a dry nanofibre matrix with or without added functional active(s) or ingredient(s), wherein the nanofibre matrix is manufactured of 100% natural biopolymers selected from polysaccharides and/or proteins and are electrospun into a dry nanofibre sheet.

29. A method to produce the nanofibre matrix, comprising the steps of: making a solution of polysaccharide and/or protein in aqueous solvents as an electrospinning solution; turning, by using electrospinning method, the electrospinning solution into dry nanofibres under high voltages; and depositing nanofibres on the collecting substrate materials.

30. The method according to claim 29, wherein the electrospinning solution is prepared by adding polysaccharide and/or protein materials sequentially into solvents under stirring for few hours, or, by preparing a polysaccharide solution and/or a protein solution separately and then mixing two solutions together.

31. The method according to any of claim 29 or claim 30, wherein functional ingredients are added into the electrospinning solution, or functional ingredients are added into the premixed polysaccharide/protein solution or functional ingredients are added into the solvents before adding polysaccharide/protein; and preferably, the pH of solution is adjusted by adding acid or base to ensure stability of functional ingredients.

32. A method to produce nanofibre matrix with functional ingredients, comprising the steps of:
dissolving polysaccharides, proteins and functional ingredients together in aqueous solvent;
turning, by using electrospinning method, solution turns into dry nanofibres under high voltages; and
depositing the nanofibres on the substrates;
wherein, the produced nanofibre matrix is a polysaccharide/protein nanofibre matrix incorporated with functional ingredients.

33. A polysaccharide/protein nanofibre mask with or without functional ingredients comprising the nanofibre matrix according to any of claims 1-28, wherein, when the mask is applied to wet skin, the nanofibers are gradually dissolved into the skin and release any actives or sits on the skin to release any actives for advanced therapeutic benefits.

34. A full beauty treatment package comprising a facial mask (whole face mask, upper face mask, lower face mask), under eye patches, forehead mask, nose mask, chin mask, cheek mask, lip mask, T-zone and or neck mask, wherein the each of the masks comprises the nanofibre matrix according to any of claims 1-28, the package is for users who have a combination type of skin, and each type of mask can be made from the same nanofibre matrix but different functional ingredient combinations to provide target different skin problems.

35. A nanofibre product made by natural and/or plant based ingredients, wherein the nanofibre product is made of the nanofibre matrix according to any of claims 1-28, and is considered as ‘edible’ cosmetic.

36. A nanofibre product made by natural and/or plant based ingredients, wherein the nanofibre product is made of the nanofibre matrix according to any of claims 1-28, and is considered as vegan products.
